(19) United States
(12) Patent Application Publication
(10) Pub. No.: US 2010/0160974 A1
(43) Pub. Date: Jun. 24, 2010

(54) Method of Bone Anchor Assembly

(75) Inventor: Thomas O. Viker, Arden Hills, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Zimmer Spine, Inc., Minneapolis, MN (US)

(21) Appl. No.: 12/341,303

(22) Filed: Dec. 22, 2008

(51) Int. Cl. A61B 17/04; A61B 19/00

(52) U.S. Cl. 606/301; 128/898; 606/305; 606/308

(57) ABSTRACT

A method of assembling a bone anchor assembly is presented. The bone anchor assembly includes a bone anchor and housing configured to receive the bone anchor and a longitudinal member. The bone anchor is inserted into a passage in the housing and material is swaged to retain the bone anchor in the housing. The material that is swaged is a portion of the housing or additional material added to the assembly.

### BACKGROUND

The disclosure relates to a bone anchor apparatus and method of assembling the apparatus which is used to retain bone portions, such as vertebrae of a spinal column, in a desired spatial relationship. Apparatus for retaining vertebrae of a spinal column in a desired spatial relationship may include a bone anchor engageable with a vertebra of the spinal column. The bone anchor connects a longitudinal member such as a rod, extendable along the spinal column to the vertebra. A housing receives the longitudinal rod and the bone anchor.

### SUMMARY

The disclosure is directed to several alternative designs and methods of manufacturing medical device structures and assemblies.

One embodiment includes a method of assembling a bone anchor system, including the steps of providing a first die having a first cavity and a positioning member extending into the first cavity, inserting a housing into the first cavity, the housing having side walls defining a bore, and the bore structured to receive a bone anchor. The method also involves inserting a bone anchor into the bore of the housing and over the positioning member, the bone anchor having a shaft, a head, and an opening in the head configured to receive the positioning member. The bone anchor is inserted such that at least a majority of the head is within the housing and the shaft extends from the housing. The method also involves swaging material around the bone anchor head thereby retaining the bone anchor in the housing.

In some embodiments, the step of swaging material includes positioning a swaging member over the bone anchor shaft and swaging the swaging member between the bone anchor head and the housing. In other embodiments, the step of swaging material includes swaging the side walls of the housing toward the bore, forming a seat for the bone anchor head.

Another embodiment is a method of assembling a bone anchor system including the steps of providing a bone anchor, the bone anchor including a head and a shaft, the head having an opening, and providing a housing having an upper portion, a lower portion, a first passage configured to receive the bone anchor, and a second passage configured to receive a longitudinal member. The method also involves providing a first die body having a positioning member configured to be received in the bone anchor head opening, providing a second die body having a first cavity configured to receive at least the upper portion of the housing and at least part of the bone anchor head, and providing a third die body having a second cavity configured to receive the bone anchor shaft and at least the lower portion of the housing, where the second cavity has inner walls in a swaging region configured to swage at least a portion of the housing inward toward the first passage. The method further involves inserting the bone anchor into the first passage in the housing through the lower portion, inserting the bone anchor and housing onto the positioning member, placing the second die body over the bone anchor shaft and housing such that the upper portion of the housing is positioned in the first cavity, placing the third die body over the bone anchor shaft and lower portion of the housing, and driving the first and third die bodies toward each other, thereby swaging at least a part of the lower portion of the housing around the anchor to secure the anchor in the housing.

An additional embodiment includes a method of assembling a bone anchor system including the steps of providing a die including a first cavity, a second cavity, and a positioning member extending into the first cavity, and providing a bone anchor assembly including a bone anchor having a head and a shaft, the head having an opening configured to receive the positioning member, and a housing configured to receive the bone anchor, the housing having side walls defining a bore, the bore structured to receive the bone anchor. The method also includes inserting the housing into the first cavity, inserting the bone anchor into the bore of the housing and onto the positioning member, such that at least a majority of the head is within the housing and the shaft extends from the housing, and swaging the side walls of the housing toward the bore, forming a seat for the bone anchor head, thereby retaining the bone anchor in the housing.

The above summary of some example embodiments is not intended to describe each disclosed embodiment or every implementation of the invention.

### BRIEF DESCRIPTION OF THE DRAWINGS

FIG. 1 is a perspective view of an un-swaged bone anchor assembly in accordance with an embodiment of the present invention;

FIG. 2 is a sectional view of the assembly of FIG. 1;

FIG. 3 is an exploded perspective view of the assembly of FIG. 1;

FIG. 4 is a sectional view of a die assembly in accordance with an embodiment of the present invention;

FIG. 5 is a sectional view of the die assembly of FIG. 4 with the top portion separated from the bottom portion;

FIG. 6 is an exploded sectional view of another embodiment of die assembly;

FIG. 7 is a sectional view of the die assembly of FIG. 5 with a bone anchor assembly positioned in the top portion of the die;

FIG. 8 is a partial sectional view of the die assembly of FIG. 4 with a bone anchor assembly positioned in the die;

FIG. 9 is a sectional view of a swaged bone anchor assembly;

FIG. 10 is a sectional view of an alternate embodiment of lower die;

FIG. 11 is a sectional view of another alternate embodiment of lower die;

FIG. 12 is a sectional view of another embodiment of swaged bone anchor assembly; and

FIG. 13 is a perspective view of a swaging member.

### DETAILED DESCRIPTION

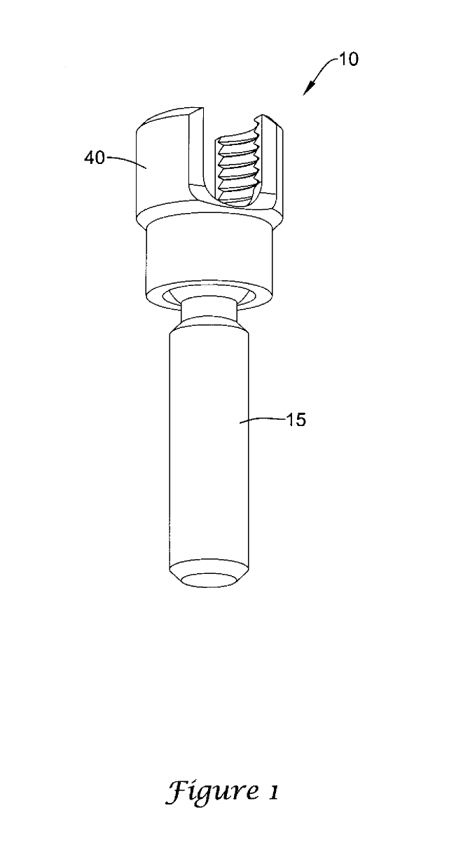

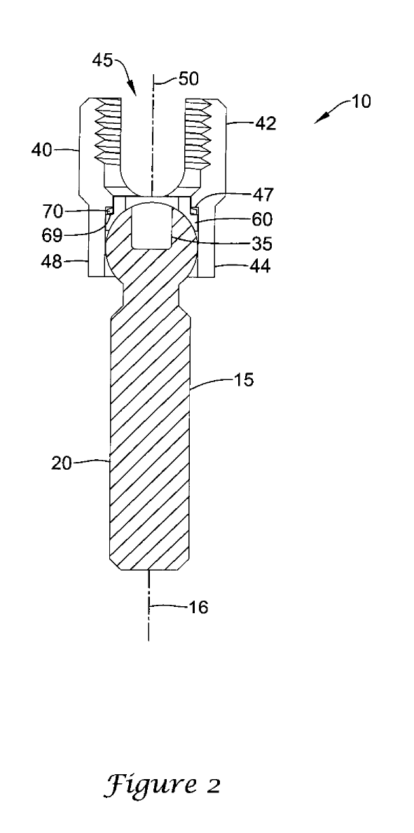

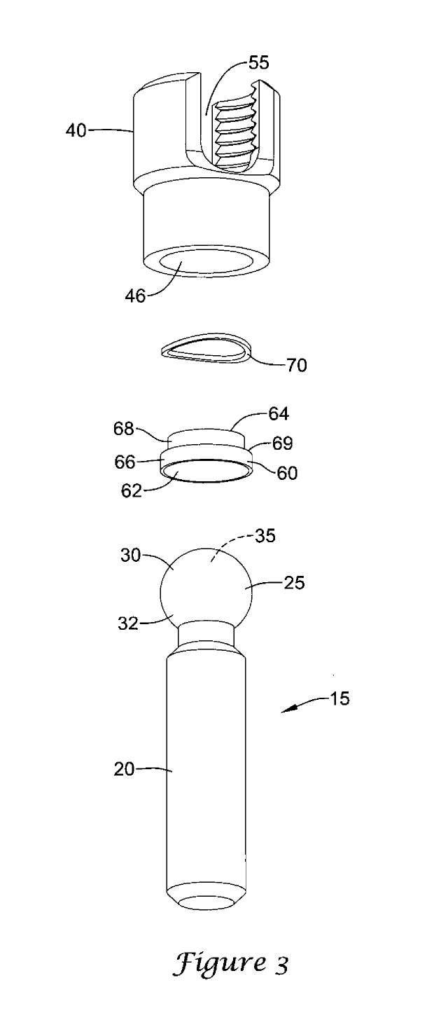

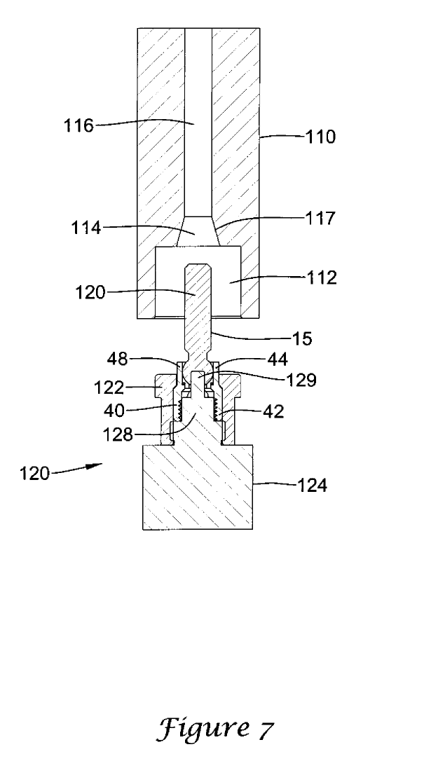

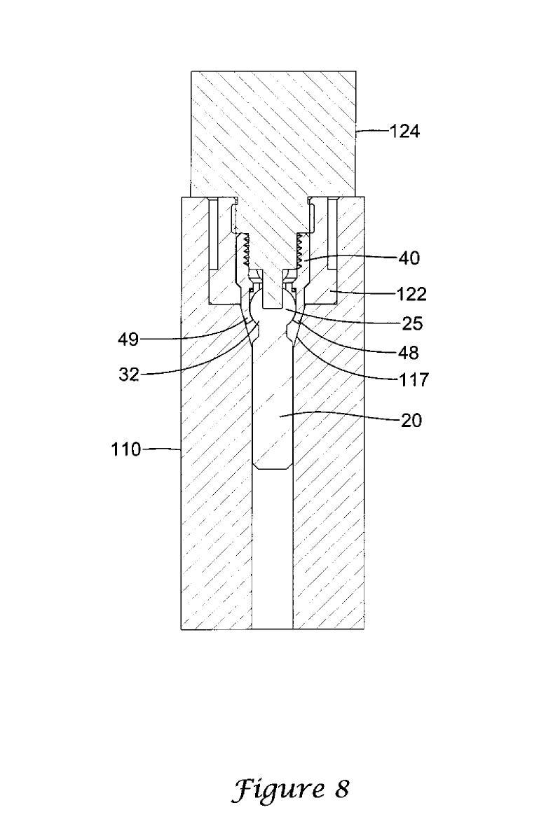

Referring now to the drawings wherein like reference numerals refer to like elements throughout the several views, FIG. 1 is a perspective view of an embodiment of a bone anchor assembly 10. The assembly 10 includes an anchor 15 made of a suitable biocompatible material. The anchor 15 includes a shaft 20 and a head 25 and has a longitudinal axis 16 (FIGS. 2, 3). In some embodiments, the anchor has a threaded shaft (not shown). The anchor shaft 20 may be elongate, as shown in FIGS. 1-3. In other embodiments, the anchor shaft is short, extending slightly below the head 25 (not shown). A head 25 of the anchor 15 is provided with an upper part spherical surface 30 and a bottom part spherical surface 32. An opening 35 is provided on the head 25 of the anchor 15. The opening 35 receives a tool (not shown) that applies force to the anchor 15 to insert the anchor into the vertebra. The opening 35 also receives a pin 129 in an upper die 120 to position the anchor 15 during swaging (FIGS. 7, 8).

The anchor 15 (FIGS. 1, 2, 3) is positioned to extend into a housing 40 that interconnects a longitudinal rod (not shown) and the anchor 15. The housing 40 has a first passage 45 with a longitudinal axis 50 that extends through the housing 40 from an upper region 42 through a lower region 44. The anchor 15 extends through an opening 46 in the bottom of the housing 40 and into the first passage 45. The housing 40 includes a circumferential edge 47 extending radially into the first passage 45 between the upper region 42 and lower region 44. A second passage 55 extends through the housing 40 transverse to the first passage 45. The second passage 55 receives the longitudinal rod.

A spacer 60 is configured to be positioned in the first passage 45 of the housing 40. The spacer 60 (FIGS. 2, 3) has a concave part spherical surface 62 that engages the upper part spherical surface 30 of the anchor 15. The spacer 60 has an opening 64 through which the tool (not shown) extends to engage the opening 35 in the anchor 15. The tool extends through the opening 64 to apply torque to the anchor 15 and connect the anchor to the vertebra.

The spacer 60 (FIG. 3) has a first outer cylindrical surface 66 with a diameter slightly smaller than the diameter of the first passage 45 in the lower region 44 of the housing 40. The spacer 60 has a second outer cylindrical surface 68 with a diameter smaller than the first outer cylindrical surface 66. A shoulder 69 connects the first and second outer cylindrical surfaces 66, 68.

A spring member 70 engages the shoulder 69. In the embodiment illustrated in FIGS. 2 and 3, the spring member 70 is a closed ring with a wave shape. In other embodiments, the spring member 70 is a split ring. When assembled, the spring member 70 is sandwiched between the circumferential edge 47 of the housing 40 and the shoulder 69 of the spacer 60 (FIG. 2). The circumferential edge 47 receives the spring member 70 and spacer 60 such that when the spacer 60 and spring member 70 are positioned on the anchor head 25, the anchor 15 is prevented from advancing into the upper region 42 of the housing 40. The spring member 70 applies an axial force to the spacer 60 to prevent relative movement between the anchor 15 and the housing 40 when the rod is disengaged from the spacer and the spacer engages the anchor.

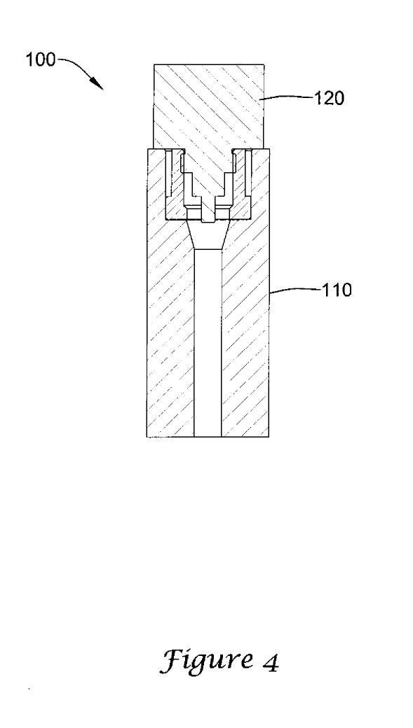

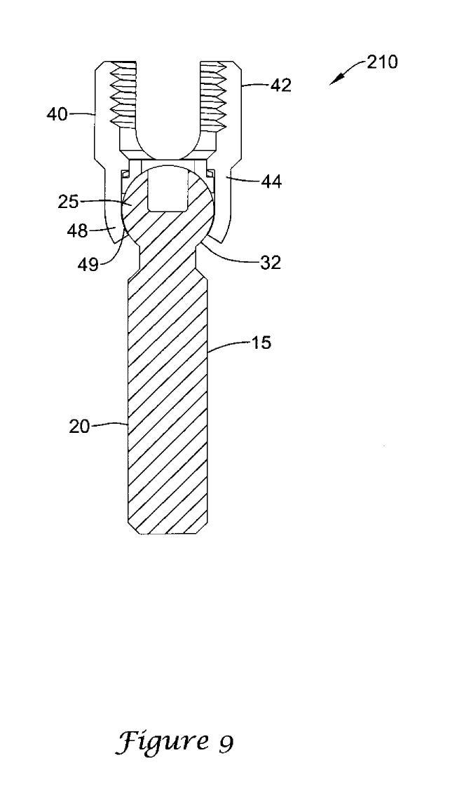

During assembly of the bone anchor assembly 10, the spring member 70, spacer 60 and anchor 15 are loaded into the housing 40 through the bottom opening 46 in the lower region 44. The spring member 70 and spacer 60 prevent the anchor 15 from advancing into the upper region 42 of the housing 40. In order to maintain the anchor 15 in the housing 40, the lower walls 48 are swaged toward the anchor 15 forming a seat 49 for the anchor head 25 (FIG. 9). The housing lower walls 48 are swaged using a die assembly 100 (FIGS. 4-6).

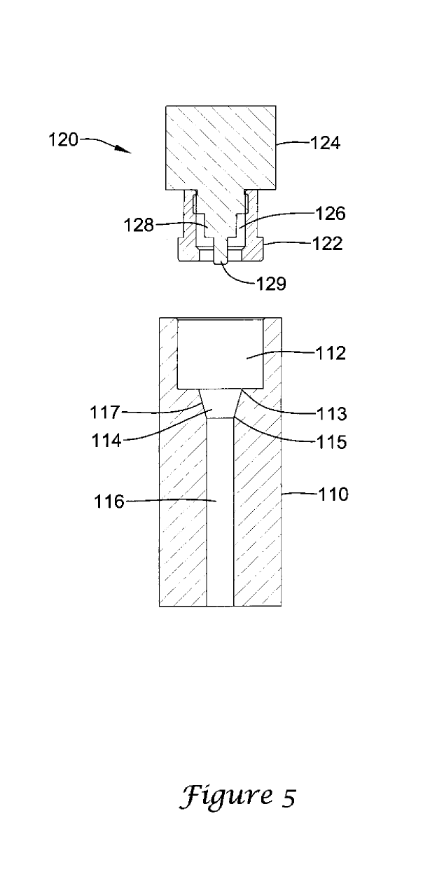

The die assembly 100 includes a lower die 110 and an upper die 120 FIGS. 4, 5). The lower die 110 includes an upper chamber 112, a middle chamber 114, and a lower chamber 116 (FIG. 5). The lower chamber 116 is configured to receive the anchor shaft 20. The middle chamber 114 is configured to receive and swage the lower region 44 of the housing 40. The upper chamber 112 is configured to receive the housing 40 and a portion of the upper die 120. The upper die 120 and upper chamber 112 position and retain the bone anchor assembly 10 during swaging.

The upper die 120 includes a base portion 124 and an extension portion 122 that is received in the upper chamber 112 of the lower die 110 (FIG. 5). The extension portion 122 includes a recess 126 configured to receive the upper region 42 and at least a portion of the lower region 44 of the housing 40. The extension portion 122 also includes a positioning member 128. The positioning member 128 includes a pin 129 configured to be received in the opening 35 in the anchor head 25 (FIG. 5).

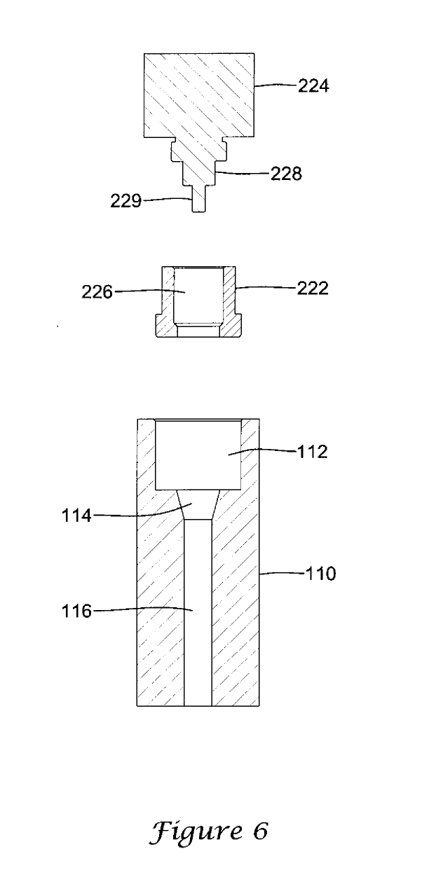

Another embodiment of upper die, illustrated in FIG. 6, includes a die base 224 and separate middle portion 222. This embodiment of upper die is configured to be used with the lower die 110 previously described. The middle portion 222 is configured to be received in the upper chamber 112 of the lower die 110. The middle portion 222 includes a recess 226 configured to receive the upper region 42 and at least a portion of the lower region 44 of the housing 40. The die base 224 includes a positioning member 228, which includes a pin 229 configured to be received in the opening 35 in the anchor head 25.

In the embodiments illustrated in FIGS. 4-8, the middle chamber 114 has an upper region 113 with an internal diameter sized to receive the housing lower region 44. The middle chamber 114 tapers inward to a lower region 115 with an internal diameter less than that of the upper region 113 and less than an outer diameter of the housing lower region 44. The taper of the middle region 114 is configured to swage the lower walls 48 of the housing 40 when the housing 40 is inserted into the die.

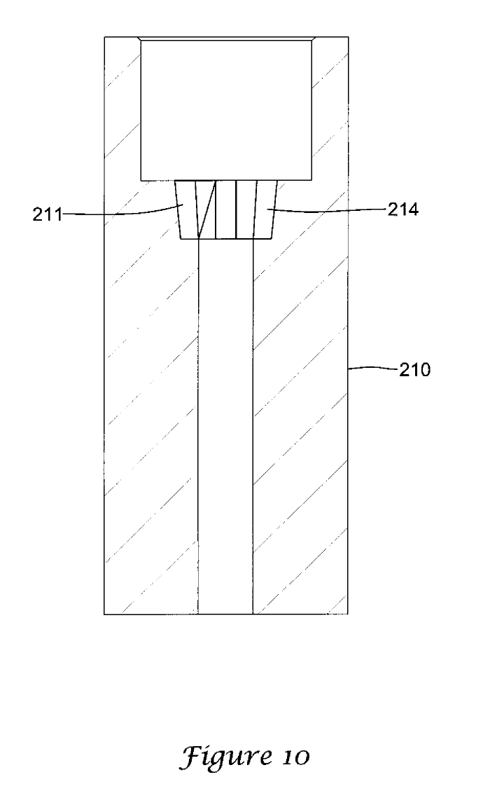

In another embodiment, illustrated in FIG. 10, the lower die 210 has a middle chamber 214 including one or more rolling members 211. The lower die 210 is rotated as it is advanced onto the bone anchor assembly 10 positioned in the upper die 120, and the rolling members 211 exert a rotational and radially compressing force onto the lower wall 48 of the housing 40, thereby swaging the housing around the anchor head 25.

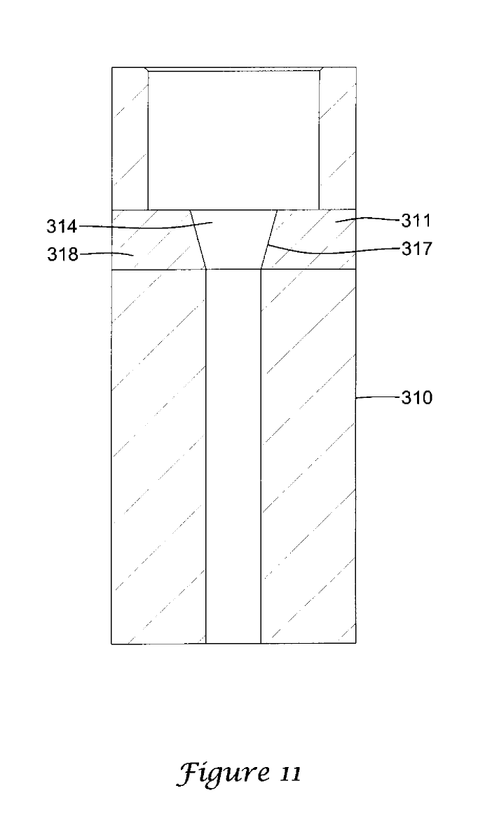

In a further embodiment, illustrated in FIG. 11, the lower die 310 includes one or more radial compression member 311 forming the middle chamber 314 having angled walls 317. A bone anchor assembly 10 is positioned in the upper die 120 and the lower die 310 is positioned over the bone anchor assembly 10. A radial compression force is exerted by the radial compression member 311 onto the lower wall 48 of the housing 40, thereby swaging the housing around the anchor head 25. In one embodiment, the radial compression member 311 includes two or more sections 318 that overlap as they are compressed thereby reducing the diameter of the middle chamber 314 to swage the lower wall 48 of the housing 40.

The method of assembling the bone anchor assembly 10 involves inserting a spring member 70, a spacer 60, and a bone anchor 15 into the bottom opening 46 in a housing 40 to form a bone anchor assembly 10 (FIGS. 1-3). Using a die assembly having a single part upper die 120, as illustrated in FIGS. 4 and 5, the upper die 120 is inverted on a work surface such that the extension portion 122 extends upward. The bone anchor assembly is then inserted into the upper die 120 with the upper region 42 of the housing positioned in the recess 126, the positioning member 128 extending into the first passage 45 of the housing 40, and the pin 129 inserted into the opening 35 in the anchor head 25 (FIG. 7). The bone anchor assembly is thus positioned and secured for swaging. The lower die 110 is then positioned over the bone anchor shaft 20 and extension portion 122 of the die. An axial force is directed onto the lower die 110, and as the lower die 110 is advanced, the angled walls 117 of the middle chamber 114 contact and swage the lower wall 48 of the housing 40 around the bottom part spherical surface 32 of the anchor head 25, forming a seat 49 (FIG. 8). The swaged bone anchor assembly 210 (FIG. 9) is removed from the die assembly 100 and used to connect a longitudinal rod to a bone portion. Alternatively, the upper die 120 may be suspended from a work surface, and the bone anchor assembly 10 and lower die 110 may be advanced upward to swage the housing wall 48 around the anchor 15.

A die assembly having a two-part upper die, such as that illustrated in FIG. 6, may also be used. The middle portion 222 of the die is positioned on the die base 224 such that the positioning member 228 extends into the recess 226. The spring member 70, spacer 50, and anchor 15 are inserted into the bottom opening 46 of the housing 40 to form the bone anchor assembly 10 as shown in FIGS. 1 and 2. The bone anchor assembly 10 is positioned in the upper die such that the upper region 42 of the housing 40 is received in the recess 226 and the positioning member 228 is received in the first passage 45 of the housing. The upper die and bone anchor assembly has a configuration similar to that illustrated in FIG. 7. The lower die 110 is then positioned over the bone anchor shaft 20 and lower region 44 of the housing, and an axial force is directed onto the lower die 110, thereby swaging the lower wall 48 of the housing around the bottom part spherical surface 32 of the anchor head 25.

The method of using a lower die 210 having one or more rolling members 211 (FIG. 10) includes the steps of assembling the spring member 70, spacer 60, and bone anchor 15 in the housing 40, and positioning the resulting bone anchor assembly 10 in the upper die 120 as described previously. The lower die 210 is then rotated as it is advanced over the bone anchor shaft 20 and lower wall 48 of the housing. When the rolling members 211 contact the lower wall 48, the rolling members 211 exert a rotational, radially compressing force onto the lower wall 48, thereby swaging the wall 48 against the anchor 15.

The method of using a lower die 310 having one or more radial compression member 311 (FIG. 11) includes the steps of assembling the spring member 70, spacer 60, and bone anchor 15 in the housing 40, and positioning the resulting bone anchor assembly 10 in the upper die 120 as described previously. The lower die 310 is then advanced completely over the bone anchor assembly and extension portion of the upper die 120. Once the lower die 310 is in position, a radial compression force is exerted on the radial compression member 311, thereby compressing the radial compression member 311 and the resulting middle chamber 314. The chamber walls 317 contact and compress the housing lower wall 48, swaging the wall 48 around the anchor head 25.

The combination of the spacer 60, spring member 70, and swaging of the housing lower wall 48 results in an anchor 15 that is movable relative to the housing 40, but cannot be removed from the housing 40. The spring member 70 urges the spacer 60 axially toward the anchor head 15 and the seat 49 of the housing 40 against the bottom part spherical surface 32 of the anchor head 25. A part spherical surface 62 of the spacer 60 frictionally engages the upper part spherical surface 30 of the anchor head 25 and the bottom part spherical surface 32 of the anchor head frictionally engages the seat 49 of the housing 40. The anchor 15 and the housing 40 are manually movable relative to each other by a surgeon when the rod is disengaged from the spacer 60 and the spring member 72 applies the axial force. The force applied by the spring member 72 may be overcome by the surgeon to move the housing 40 relative to the anchor 15. It is contemplated that any compressible member could be used to apply the force to the anchor 15 to prevent relative movement between the anchor and the housing 40 when the rod is disengaged from the spacer 60. Accordingly, the anchor 15 is universally pivotable relative to the housing 40 so that the longitudinal axis 16 of the anchor 15 is positionable in any one of a plurality of angular positions relative to the longitudinal axis 50 of the passage 45.

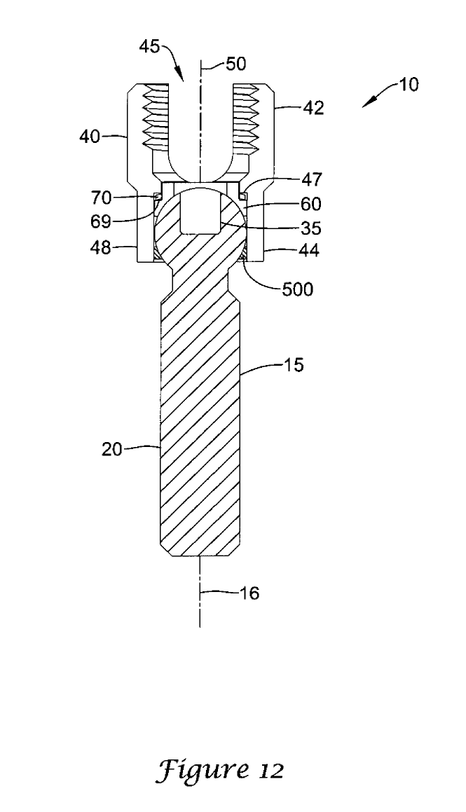

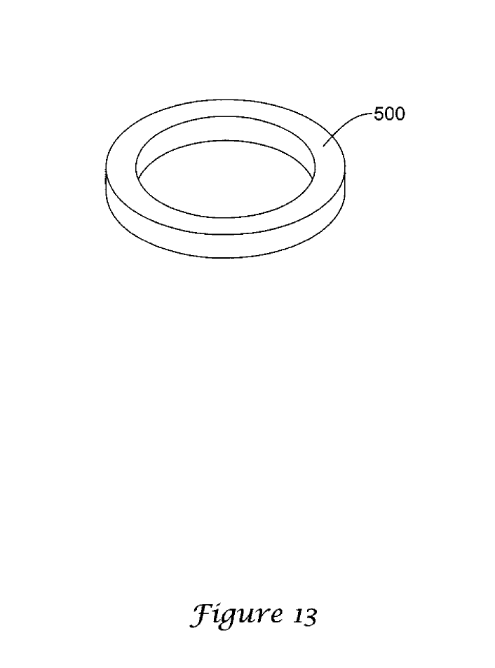

In another embodiment, a swaging member 500 is inserted over the anchor shaft 20 after the anchor 15 is positioned in the housing 40 (FIG. 12). The swaging member 500 is sized to fit between the bottom part spherical surface 32 of the anchor head 25 and the lower wall 48 of the housing 40. The swaging member 500 is swaged into the space between the bottom surface 32 of the anchor head 25 and the lower wall 48 of the housing 40 to prevent the anchor 15 from being removed from the housing 40. In some embodiments, the swaging member 500 is a ring of material having a hardness less than that of the anchor 15 and housing 40 (FIG. 13). The swaging member 500 may be a metal, polymer, or other material capable of being swaged to retain the anchor 15 in the housing 40.

Numerous characteristics and advantages of the invention covered by this document have been set forth in the foregoing description. It will be understood, however, that this disclosure is, in many respects, only illustrative. Changes may be made in details, particularly in matters of shape, size and ordering of steps without exceeding the scope of the invention. The invention's scope is, of course, defined in the language in which the appended claims are expressed.

What is claimed is:

1. A method of assembling a bone anchor system comprising:
providing a first die having a first cavity and a positioning member extending into the first cavity;
inserting a housing into the first cavity, the housing having side walls defining a bore, the bore structured to receive a bone anchor;
inserting a bone anchor into the bore of the housing and over the positioning member, the bone anchor having a shaft, a head, and an opening in the head configured to receive the positioning member, wherein the bone anchor is inserted such that at least a majority of the head is within the housing and the shaft extends from the housing; and
swaging material around the bone anchor head thereby retaining the bone anchor in the housing.

2. The method of claim 1, wherein the step of swaging includes positioning a swaging member over the bone anchor shaft and swaging the swaging member between the bone anchor head and the housing.

3. The method of claim 1, wherein the step of swaging includes swaging the side walls of the housing toward the bore, forming a seat for the bone anchor head.

4. The method of claim 3, wherein the step of swaging is performed by applying an axial force on the side walls to move the side walls radially toward the shaft, by placing a second die over the bone anchor; the second die having a second cavity structured to receive the shaft and at least part of the housing, the second cavity having inner walls structured to apply an axial force on the housing side walls when the second die is placed over the housing.

5. The method of claim 4, wherein the second cavity inner walls are angled such that placing the second die over the bone anchor and housing causes the side walls of the housing to move radially thereby capturing the bone anchor head in the housing.

6. The method of claim 3, wherein the step of swaging is performed by applying a rotational and an axial force on the side walls to move the side walls radially toward the shaft, by placing a second die over the bone anchor and rotating the second die as it is advanced onto the bone anchor shaft and the first die, the second die having a second cavity defined by rolling members, the second cavity structure to receive the shaft and at least part of the housing, the rolling members structured and angled, wherein when the second die is rotated and advanced onto the bone anchor and housing, the rolling members contact and swage the housing side walls against the bone anchor.

7. The method of claim 3, wherein the step of swaging is performed by applying a radially compression force on the side walls to move the side walls radially toward the shaft.

8. The method of claim 7, wherein the step of applying a radially compression force is achieved by placing a second die over the bone anchor and radially compressing the housing side walls; wherein the second die has a second cavity structured to receive the shaft and at least part of the housing, the second cavity defined by one or more radial compression members structured to apply a radial compression force on the housing side walls when the second die is placed over the housing.

9. The method of claim 1, wherein after the step of inserting a housing, the method further includes inserting a retainer into the bore, the retainer structured to prevent the bone anchor head from extending completely through the housing.

10. The method of claim 9, wherein the bore includes an engagement member and the step of inserting a retainer includes inserting the retainer such that the retainer engages the engagement member.

11. The method of claim 10, wherein the engagement member is a recess and the retainer is a ring.

12. A method of assembling a bone anchor system comprising:
providing a bone anchor, the bone anchor including a head and a shaft, the head having an opening;
providing a housing, the housing having an upper portion, a lower portion, a first passage configured to receive the bone anchor, and a second passage configured to receive a longitudinal member;
providing a first die body having a positioning member configured to be received in the bone anchor head opening;
providing a second die body having a first cavity configured to receive at least the upper portion of the housing and at least part of the bone anchor head;
providing a third die body having a second cavity configured to receive the bone anchor shaft and at least the lower portion of the housing, the second cavity having inner walls in a swaging region configured to swage at least a portion of the housing inward toward the first passage;
inserting the bone anchor into the first passage in the housing through the lower portion;
inserting the bone anchor and housing onto the positioning member;
placing the second die body over the bone anchor shaft and housing such that the upper portion of the housing is positioned in the first cavity;
placing the third die body over the bone anchor shaft and lower portion of the housing; and
driving the first and third die bodies toward each other, thereby swaging at least a part of the lower portion of the housing around the anchor to secure the anchor in the housing.

13. The method of claim 12, wherein the second cavity has angled walls that contact the lower portion of the housing when the first and third die bodies are driven toward each other, thereby swaging the lower portion of the housing around the anchor head.

14. The method of claim 13, wherein before inserting the bone anchor into the housing, the method further comprises the step of inserting a spacer into the first passage of the housing, the spacer applying an axial force toward the anchor head to create a frictional engagement between the anchor head and an inner surface of the housing.

15. The method of claim 14, wherein before inserting the spacer, the method further comprises the step of inserting a spring member into the first passage of the housing, the spring member compressing to apply an axial force to the spacer and urging the spacer axially toward the anchor head to prevent relative movement between the anchor and the housing.

16. The method of claim 12, wherein the step of driving the first and third die bodies toward each other includes rotating the third die body as the cavity walls contact the lower portion of the housing, thereby moving the lower portion of the housing radially toward the shaft.

17. The method of claim 16, wherein the second cavity includes roller members structured to apply a rotational radial force on the lower portion of the housing when the third die body is rotated over the housing.

18. A method of assembling a bone anchor system comprising:
providing a die including a first cavity, a second cavity, and a positioning member extending into the first cavity;
providing a bone anchor assembly including a bone anchor having a head and a shaft, the head having an opening configured to receive the positioning member, and a housing configured to receive the bone anchor, the housing having side walls defining a bore, the bore structured to receive the bone anchor;
inserting the housing into the first cavity;
inserting the bone anchor into the bore of the housing and onto the positioning member, such that at least a majority of the head is within the housing and the shaft extends from the housing;
swaging the side walls of the housing toward the bore, forming a seat for the bone anchor head, thereby retaining the bone anchor in the housing.

19. The method of claim 18, wherein the die includes an upper die forming the first cavity, and a lower die forming the second cavity; wherein the upper die includes a die base having the positioning member configured to extend into the bore of the housing, and a die middle portion forming the first cavity; wherein the step of inserting the housing into the first cavity involves positioning the middle portion over the housing positioning member such that the first cavity is formed between an inner wall of the middle portion and the positioning member, and inserting the housing into the cavity.

20. The method of claim 19, wherein the second cavity in the lower die includes an upper chamber configured to receive the die middle portion, a middle chamber configured to receive and swage the side walls of the housing, and a lower chamber configured to receive the bone anchor shaft.
